(19) United States
(12) Patent Application Publication
(10) Pub. No.: US 2012/0289744 A1
(43) Pub. Date: Nov. 15, 2012

(54) PROCESS FOR PREPARING OPTICALLY PURE MILNACIPRAN AND ITS PHARMACEUTICALLY ACCEPTABLE SALTS

(75) Inventors: Ganesh Gurpur Pai, Thane (IN); Arun Kanti Mandal, Mumbai (IN); Kamlesh Jayantilal Ranbhan, Thane (IN); Vikram Sarjerao Jagtap, Mumbai (IN); Dayaghan Gangadhar Patil, Kalyan (IN); Yuvraj Kashinath Zunjarrao, Thane (IN)

(73) Assignee: ARCH PHARMALABS LIMITED, Mumbai (IN)

(21) Appl. No.: 13/574,775

(22) PCT Filed: Dec. 20, 2010
(86) PCT No.: PCT/IN2010/000826

§ 371 Date: Jul. 23, 2012

(30) Foreign Application Priority Data
Nov. 3, 2010 (IN) 3054/MUM/2010

(51) Int. Cl. C07C 231/16; C07C 237/20

(52) U.S. Cl. 564/164

(57) ABSTRACT

The present invention relates to an improved and commercially, viable process for the resolution of racemic cis milnacipran of formula I and its pharmaceutically acceptable salts of formula II. The present invention comprises using racemic cis milnacipran or its pharmaceutically acceptable salts as starting material, a low cost and commercially available resolving agent of formula III and industrially safe and economically low cost material such as water as a solvent. The said process results into optical isomers of racemic cis milnacipran having excellent optical purity without involving multiple crystallization steps. The present invention also comprises the concept of green chemistry as the invention works well with water as a solvent thereby minimizing the use of any other solvent. (Formular I and II should be inserted here) Wherein X is anion selected from Cl, Br, I, HSO₄, Phosphate or organic acid (Formular III should be inserted here) *represent asymmetric centre Compound of formula III represent mandelic acid and its derivatives.

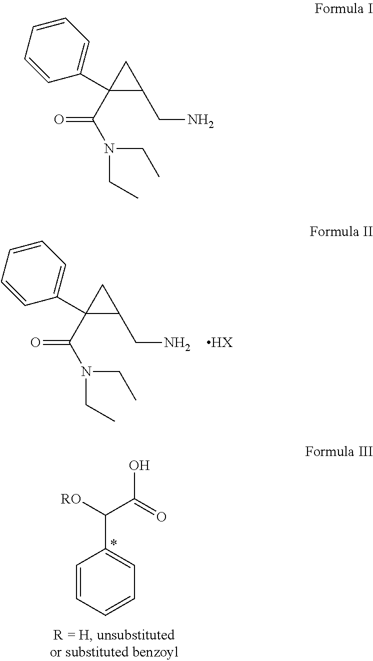

### TECHNICAL FIELD OF THE INVENTION

The present invention relates to an improved and commercially viable process for the resolution of racemic cis milnacipran of formula I and its pharmaceutically acceptable salts of formula II. The present invention comprises using racemic cis milnacipran or its pharmaceutically acceptable salts as starting material, a low cost and commercially available resolving agent of formula III and industrially safe and economically low cost material such as water as a solvent. The said process results into optical isomers of racemic cis milnacipran having excellent optical purity avoiding multiple crystallizations. The present invention also comprises the concept of green chemistry as the invention works well with water as a solvent thereby minimizing the use of any other solvent.

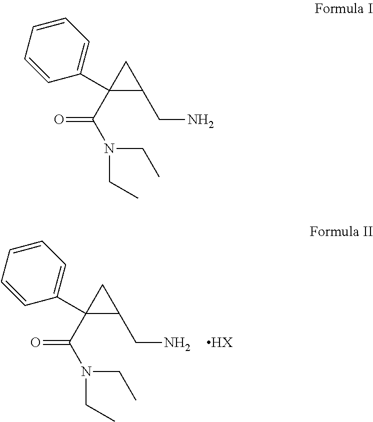

Wherein X is anion selected from Cl, Br, I, HSO₄, Phosphate or organic acid

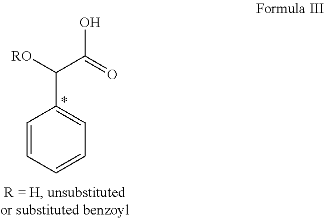

*represent asymmetric centre

Compound of formula III represent mandelic acid and its derivatives.

### BACKGROUND OF THE INVENTION

Racemic milnacipran chemically named as 1-phenyl-2-(aminomethyl)cyclopropane-N,N-diethyl carboxamide) was first approved for the treatment of major depressive episodes in France in December 1996. It is currently marketed (as Ixel) for this indication in over 45 countries worldwide including several European countries such as Austria, Bulgaria, Finland, France, Portugal, and Russia. It is also available in Japan (as Toledomin) and Mexico (as Dalcipran). Cypress Bioscience bought the exclusive rights for approval and marketing of the drug for any purpose in the United States and Canada in 2003 from the inventor Pierre Fabre. It is reported that said drug is also used to treat fatigue, pain, fibromyalgia, Irritable bowel syndrome and the like. At present, it is mostly sold in the form of racemic cis milnacipran hydrochloride. Milnacipran belongs to dual inhibitors of serotonin and norepinephrine reuptake (SNRI), which is the fourth generation antidepressant and can inhibit both serotonin and norepinephrine reuptakes, with similar action strength. It is mainly useful to treat depression, especially major depression. Currently 22 countries have approved racemic cis milnacipran for treating depression. Cis milnacipran (Z(±)-2-(amino methyl)-N,N-diethyl-1-phenyl cyclopropane carboxamide), a molecule synthesized at the PIERRE FABRE MEDICAMENT Research Centre (Castres, France), also called as TN-912, dalcipran, minalcipran, midalcipran or midalipran is known to be a dual inhibitor of serotonin (5-HT) and norepinephrine (NE) reuptake. Dual inhibitors of serotonin (5-HT) and norepinephrine (NE) reuptake correspond to a well-known class of antidepressant agents which selectively inhibit reuptake of both serotonin and norepinephrine. By way of example, venlafaxine and duloxetine are also dual inhibitors of serotonin and norepinephrine. Studies have shown that the ratio of norepinephrine reuptake inhibition to serotonin reuptake inhibition by cis milnacipran is approximately 2:1 (Moret et al., 1985 Neuropharmacology 24(12): 1211-1219; Palmier et al., 1989, Eur J Clin Pharmacol 37: 235-238).

In January 2009 the U.S. Food and Drug Administration (FDA) approved racemic cis milnacipran (under the brand name Savella) for the treatment of fibromyalgia, making it the third medication approved for this purpose in the United States. Fibromyalgia, which is estimated to affect from 2-4% of the population in the US, is a complex syndrome associated with chronic widespread musculoskeletal pain and a reduced pain threshold, with hyperalgesia and allodynia (pain-related behavior in response to normally innocuous stimuli). Some associated clinical features include fatigue, depression and other mood disorders, anxiety, sleep disturbances, headache (including migraine), changes in bowel habits (including irritable bowel syndrome), diffuse abdominal pain, and urinary frequency.

The study of racemic cis milnacipran has revealed that the medication containing cis milnacipran does not interact with other medications as it has lower ability of plasma protein-binding, moreover cis milnacipran's half-life is relatively shorter, it has an advantage of no residual effect after treatment, and therefore it has fine tolerance and security. Therefore it has acquired a lot of importance in the present scenario.

In 1992, a resolution had been approved by American Food and Drug Administration (FDA) and The European Committee for Proprietary Medicinal Products, which encouraged that drugs with chiral center should be in optically pure form for marketing authorization; in 1996, a project had been proposed by FDA that drugs with chiral center must be in optically pure form when it is applying for marketing authorization. There are two chiral centers in the molecular structure of milnacipran; there should be two groups of enantiomers and therefore four compounds in theory. Due to the molecular configuration, the cis-isomer is the main synthetic product, that exists in two forms of optical enantiomers: the dextrogyral enantiomer of cis-milnacipran hydrochloride Z-(1S,2R) of formula IV chemically named as Z-(1S,2R)-2-(amino methyl)-N,N-diethyl-1-phenyl cyclopropane carboxamide and the levogyral enantiomer of cis-milnacipran hydrochloride Z-(1R,2S) of formula V chemically named as Z-(1R,2S)-2-(amino methyl)-N,N-diethyl-1-phenyl cyclopropane carboxamide. In its hydrochloride form, cis milnacipran (also called F2207) is currently marketed as Iexel in the form of a racemic mixture as a serotoninergic and norepinephrinergic antidepressant agent. F2695 and F2696 represent the dextrogyral and levogyral enantiomers respectively of cis milnacipran hydrochloride (F2207) and are represented as mirror images as shown below:

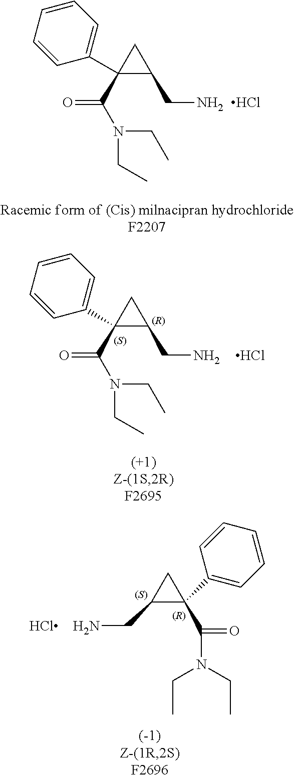

The molecular structural formulae of the two chiral enantiomers of cis-milnacipran and its pharmaceutically acceptable salts are as follows:

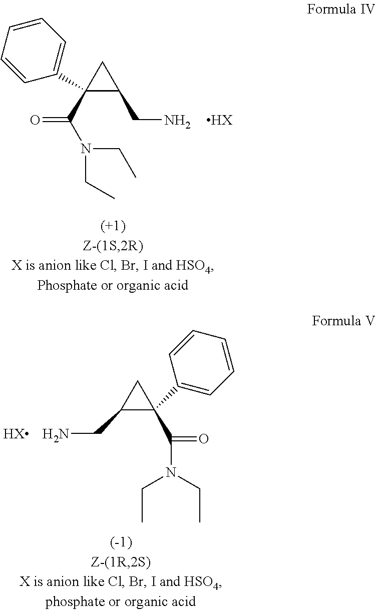

However the prior art does not disclose possibility of representing the cis isomer of the milnacipran hydrochloride as given below:

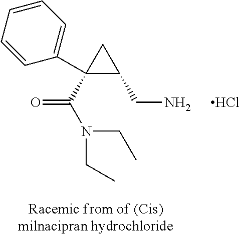

Hence another possible two Z conventions for the above structure can be represented as follows which will be mirror images of each other.

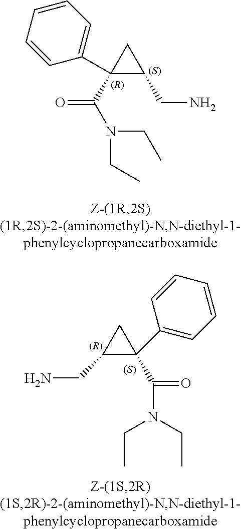

However, in the present text racemic cis milnacipran is represented by formula I and its hydrochloride by formula II, wherein X is Cl. In the present invention compound of formula I represent F2207.

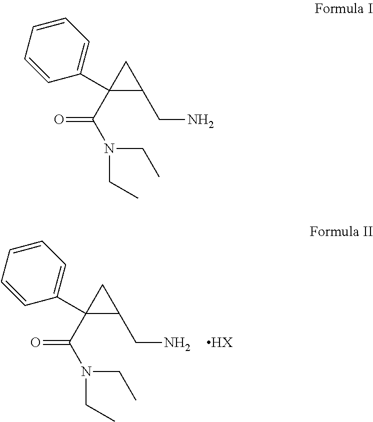

Wherein X is anion selected from Cl, Br, I, HSO₄, Phosphate or organic acid

The processes reported therein in the prior art for preparing optically pure cis milnacipran are mostly the asymmetric synthesis methods. Cis milnacipran and its method of preparation are described in U.S. Pat. No. 4,478,836. The said patent also describes the use of cis milnacipran for the treatment of disorders of the central nervous system, in particular depression. WO01/26623 describes the use of milnacipran in association with phenylalanine and tyrosine in indications such as the treatment of fatigue, syndromes associated with pain, chronic fatigue syndrome, fibromyalgia, and irritable bowel syndrome. WO01/62236 describes a composition containing milnacipran in association with one or several antimuscarinic agents for a large number of indications including depression. WO97/35574 describes a pharmaceutical composition containing milnacipran and idazoxan as an associated product for use simultaneously, separately or staggered in time to treat depression and its various forms, as well as disorders in which antidepressants are used. Cis-milnacipran is also indicated for use in the treatment of urinary incontinence (FR 2759290).

US 2004/0162334, US 20060014837 and CN 1699332A disclose that the dextrogyral enantiomer of cis milnacipran hydrochloride had activity which was significantly higher than racemic cis milnacipran, with less risk of cardiovascular disturbances and tissue and organ organic toxicity.

Bonnaud et al. Journal of Chromatography, vol 318:398-403, 1985 discloses the resolution of racemic γ-butyrolactone (1-phenyl-3-oxa-bicyclo-[3.1.0]hexane-2-ketone) by R-(+)-phenyl ethylamine. Optically active γ-butyrrolactone was separated by chiral stationary phase method (HPLC). γ-butyrrolactone is an intermediate for the preparation of cis milnacipran. The following schematic representation shows the resolution of racemic γ-butyrolactone of formula L-I to get the corresponding enantiomers which can be converted into respective enantiomer of cis milnacipran.

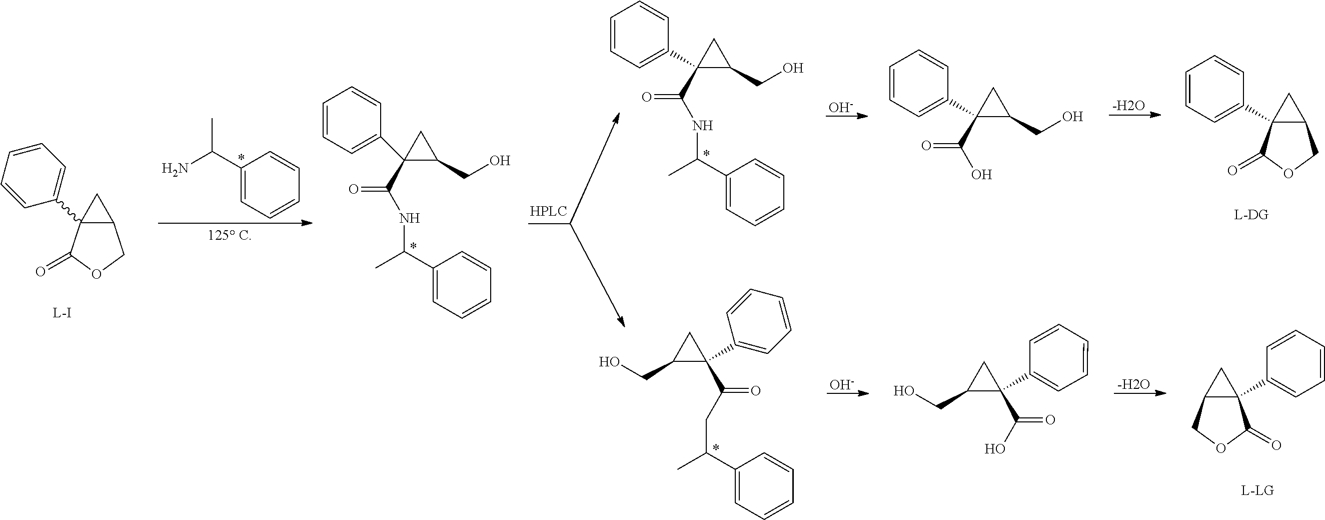

Shuto et al., Tetrahedron letters, 1996 Vol. 37: 641-644) disclose use of optically active epichlorohydrin and benzacetonitrile as starting materials. Optically fused γ-butyrrolactone of formula L-I was obtained by hydrolysis after nucleophilic substitution is performed twice in the presence of sodium amide. Then the ring of γ-butyrrolactone was opened to liberate hydroxyl group, substituted by azide group, reduced to give (+) and (−)-cis milnacipran. The following schematic representation shows the preparation of optically active γ-butyrolactone (1-phenyl-3-oxa-bicyclo [3.1.0]hexane-2-ketone) as key intermediates those can be converted into respective cis milnacipran molecules. The said reference also discloses that dextrogyral cyano compound of formula-DGN when subjected to alkaline hydrolysis using NaNH₂ as base and benzene as solvent at room temperature resulted into most desirable dextrogyral(1S,2R)-γ-butyrolactone with 96% e.e. and 67% yield.

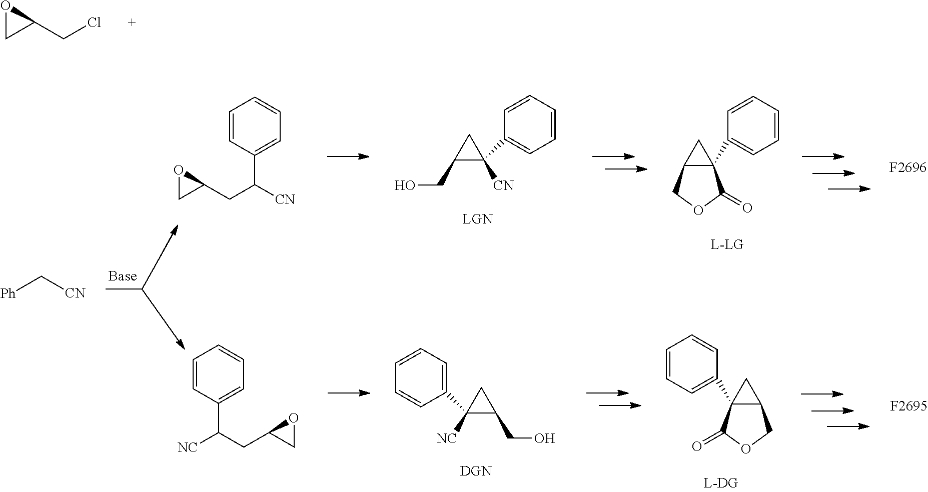

Grard et al., Electrophoresis, 2000, 21:3028-3034 discloses the separation of racemic cis milnacipran to give optically pure milnacipran by high performance capillary electrophoresis chirality method.

Doyle and Hu discloses phenylacetic acid as starting material; optically pure cis milnacipran can be obtained after asymmetric catalysis (Doyle and Hu 2001, Advanced Synthesis and Catalysis Vol. 343:299-302). Both the asymmetric synthesis method and the chromatography method can give optically pure milnacipran with higher e.e. value (enantiomeric excess), but with complex operations and high cost.

U.S. Pat. No. 7,005,452 discloses the use of a mixture of enantiomers enriched in the dextrogyral enantiomer of cis milnacipran as well as their pharmaceuticallγ-acceptable salts, for the preparation of a drug intended to prevent or to treat disorders that can be managed by double inhibition of serotonin (5-HT) and norepinephrine (NE) reuptake, while limiting the risks of cardiovascular disturbances and/or organ and/or tissue toxicity.

US2010/0016636 discloses a process for the preparation of optically pure cis milnacipran and their pharmaceutically acceptable salts comprising racemic cis-milnacipran as starting material and tartaric acid derivatives and their compositions as resolving agents. Paragraph 0011, page No, 2 discloses general structure (given below) of resolving agents (termed as split reagent) which represent tartaric acid derivatives.

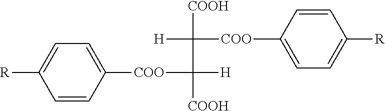

when R is H, it is di-benzoyl tartaric acid
when R is CH₃, it is di-p-toluoyl tartaric acid
when R is OCH₃, it is di-p-methoxybenzoyl tartaric acid

Paragraph 0012 of US2010/0016636 clearly states that resolving agents are composed of di-p-toluoyl tartaric acid, di-p-methoxybenzoyl tartaric acid and di-benzoyl tartaric acid. However there is no disclosure or teaching in the said reference for the use of tartaric acid as such as a resolving agent for the resolution of racemic cis milnacipran. Inventors of the present invention have also tried and confirmed that tartaric acid as such does not work as a resolving agent for the resolution of racemic cis milnacipran. Drawbacks associated with prior art are the use of expensive tartaric acid derivatives such as di-p-toluoyl tartaric acid, di-p-methoxybenzoyl tartaric acid and di-benzoyl tartaric acid as resolving agent for the resolution of cis racemic mixture to obtain optically active cis milnacipran. This is based on the fact that the tartaric acid is dibasic in nature, therefore, it is apparent that preparation of the said di-p-toluoyl tartaric acid, di-p-methoxybenzoyl tartaric acid and di-benzoyl tartaric acid derivatives will require two moles of respective reactants, thereby indicating the sufficiency of requiring 0.5 mol tartaric acid derivative for the resolution of 1 mol of racemic cis milnacipran. However it is evident from the examples disclosed in the US2010/0016636 that the minimum stoichiometric ratio used therein is 1:1 for milnacipran base generated insitu i.e. without isolation is considered to be 100%. Other drawbacks are non use of tartaric acid as such as resolving agent, multiple number (at least three) of crystallizations to achieve the required optical purity thereby reducing the yield drastically, less optical purity, use of azide and therefore reduction of azide group, use of NaNH₂ as base and benzene as solvent which itself is carcinogenic and is restricted as per the ICH guidelines for the selective isolation of dexrogyral isomer.

Prior art discloses generally asymmetric processes for the preparation of optically active pure cis milnacipran. US2010/0016636 only discloses the resolution of racemic mixture of cis milnacipran comprising use of expensive tartaric acid derivatives such as of di-p-toluoyl tartaric acid, di-p-methoxybenzoyl tartaric acid and di-benzoyl tartaric acid as resolving agents without any indication or teaching that tartaric acid as such can also be used as a resolving agent. US2010/0016636 discloses the resolving agents represented by the compound of formula A given below which can be used for the resolution of racemic milnacipran into optically active milnacipran.

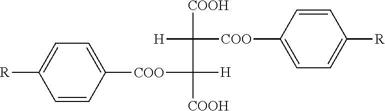

when R is H, it is di-benzoyl tartaric acid
when R is CH₃, it is di-p-toluoyl tartaric acid
when R is OCH₃, it is di-p-methoxybenzoyl tartaric acid

There is no indication or teaching which can motivate a person skilled in the art that the tartaric acid as such without its benzoyl ester can be used as a resolving agent. The same has been confirmed by the inventors of the present invention.

In view of shortcomings in the prior art there is a need for an industrially safe and commercially viable process for the preparation of optically pure cis milnacipran or cis milnacipran enriched with specified configuration and their pharmaceutically acceptable salts in high yields with higher optical purity.

Inventors of the present invention, while working on the methods disclosed in US2010/0016636 found that at least three crystallizations are needed to achieve required purity of enantiomer of cis milnacipran. However our inventors came with the finding that the use of proposed resolving agent produces excellent yield and excellent optical purity in first stroke itself while isolating the product. Another highlighting feature of the present invention is the use of water as solvent for the resolution of racemic cis milnacipran yielding high and high optical purity therefore only one crystallization is sufficient to get the required pharmaceutically acceptable purity.

Inventors of the present invention have proposed a novel process that comprises the use of racemic cis milnacipran as a starting material and low cost commercially available monobasic resolving agent of formula III.

There is neither any teaching nor any motivation in the prior art for using water as a solvent for the resolution of racemic cis milnacipran. In the present invention water is used as a solvent which not only gives excellent optical purity of about 98% but also higher yield in the range of about 88-90%.

### OBJECT OF THE INVENTION

The object of the present invention is to provide an efficient process for the preparation of optically pure cis milnacipran and pharmaceutically acceptable salts thereof with higher optical purity and higher yield. The process disclosed herein is industrially safe, economical, and simple to practice to obtain two kinds of configurationally optically pure cis milnacipran.

First aspect of the invention is to provide an efficient process for the preparation of optically pure dextrogyral enantiomer of cis-milnacipran hydrochloride Z-(1S,2R) of formula IV wherein X is Cl, chemically named as Z-(1S,2R)-2-(amino methyl)-N,N-diethyl-1-phenyl cyclopropane carboxamide hydrochloride with higher optical purity and higher yield and other pharmaceutically acceptable salts thereof.

Second aspect of the invention is to provide an efficient process for the preparation of optically pure levogyral enantiomer of cis-milnacipran hydrochloride Z-(1R,2S) of formula V wherein X is Cl; chemically named as Z-(1R,2S)-2-(amino methyl)-N,N-diethyl-1-phenyl cyclopropane carboxamide hydrochloride with higher optical purity and higher yield and other pharmaceutically acceptable salts thereof which can be used as reference marker and reference standard in analytical development during the quantitative analysis of (1S,2R)-cis-milnacipran or pharmaceutical salts thereof.

Third aspect of the invention is to provide dextrogyral enantiomer of cis-milnacipran hydrochloride Z-(1S,2R) of formula IV wherein X is Cl; chemically named as Z-(1S,2R)-2-(amino methyl)-N,N-diethyl-1-phenyl cyclopropane carboxamide hydrochloride with optical purity of about +98% with yield of about 89%.

Fourth aspect of the invention is to provide dextrogyral enantiomer of cis-milnacipran hydrochloride Z-(1S,2R) of formula IV wherein X is Cl; chemically named as Z-(1S,2R)-2-(amino methyl)-N,N-diethyl-1-phenyl cyclopropane carboxamide hydrochloride with optical purity of about +99% with yield of about 75%-80%.

Fifth aspect of the invention is to, provide levogyral enantiomer of cis-milnacipran hydrochloride Z-(1R,2S) of formula V wherein X is Cl; chemically named as Z-(1R,2S)-2-(amino methyl)-N,N-diethyl-1-phenyl cyclopropane carboxamide hydrochloride with optical purity of about 96%.

Sixth aspect of the invention is to use compound of formula III as resolving agent preferably when R is H.

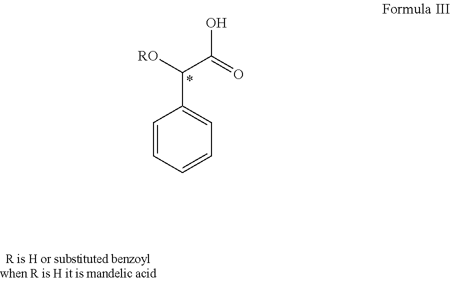

*represent asymmetric centre

Compound of formula III represent mandelic acid and its derivatives. Seventh aspect of the invention is to provide a novel and crystalline compound of the formula A

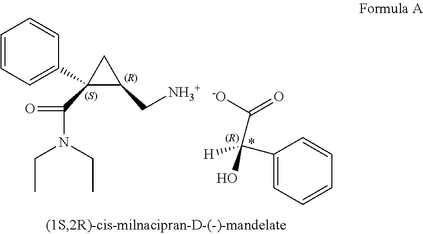

Eighth aspect of the invention is to provide a novel and crystalline compound of the formula B

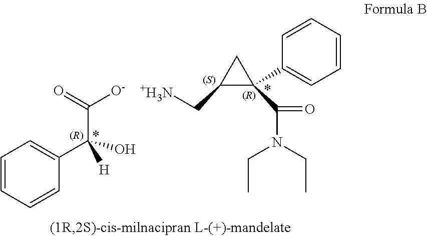

Ninth aspect of the invention is to provide a novel process for the preparation of dextrogyral enantiomer of cis-milnacipran hydrochloride Z-(1S,2R) of formula IV wherein X is Cl; chemically named as Z-(1S,2R)-2-(amino methyl)-N,N-diethyl-1-phenyl cyclopropane carboxamide hydrochloride using D-(−)-mandelic acid as a resolving agent and organic solvent.

Tenth aspect of the invention is to provide a process for the preparation of dextrogyral enantiomer of cis-milnacipran hydrochloride Z-(1S,2R) of formula IV wherein X is Cl; chemically named as Z-(1S,2R)-2-(amino methyl)-N,N-diethyl-1-phenyl cyclopropane carboxamide hydrochloride and other salts thereof using D-(−)-mandelic acid as a resolving agent and water as a solvent,

Eleventh aspect of the invention is to provide a novel process for the preparation of levogyral enantiomer of cis-milnacipran hydrochloride Z-(1R,2S) of formula V wherein X is Cl; chemically named as Z-(1R,2S)-2-(amino methyl)-N,N-diethyl-1-phenyl cyclopropane carboxamide hydrochloride using L-(+)-mandelic acid as a resolving agent and an organic solvent.

Twelfth aspect of the invention is to provide a process for the preparation of levogyral enantiomer of cis-milnacipran hydrochloride Z-(1R,2S) of formula V wherein X is Cl; chemically named as Z-(1R,2S)-2-(amino methyl)-N,N-diethyl-1-phenyl cyclopropane carboxamide hydrochloride and other salts thereof using L-(+)-mandelic acid as resolving agent and water as a solvent.

The object of the present invention is achieved by technical solution as follows:

The present invention discloses a process for the resolution of racemic cis isomer of milnacipran or its salt comprising using optically pure resolving agent of formula III preferably wherein R is H in the ratio of 1:0.5-1.5 in a solvent. In case when cis milnacipran salts like hydro halide preferably cis milnacipran hydrochloride is used as a starting material it is converted into milnacipran base by the general process disclosed in the prior art. The process comprises dissolution of racemic cis-milnacipran base in a solvent till a clear solution is obtained followed by reacting with optically pure resolving agent of formula III preferably when R is H, heat if necessary followed by cooling till cis milnacipran mandelate is precipitated. The salt so obtained is filtered off and washed. The same salt is taken in a mixture of organic solvent and water followed by the addition of alkali to obtain substantially optically enriched cis milnacipran isomer with optical purity of about 98 to about 99.5%.

### ADVANTAGES OVER THE PRIOR ART

1. Use of simple and low cost commercially available resolving agents thereby reducing overall costing of the product.
2. Higher yield and higher optical purity.
3. Use of water as solvent.
4. Avoiding multiple numbers of crystallizations to achieve required purity.
5. Avoiding chromatographic separations.
6. The process is low cost, easy to operate, suitable for industrial scale production.
7. The process affords two kinds of chiral enantiomers/optical isomers of cis milnacipran.
8. Only one crystallization is sufficient to achieve the required purity of required cis milnacipran enantiomer.

### SUMMARY OF THE INVENTION

The present invention discloses an efficient, novel and commercially viable process for resolution of racemic cis milnacipran. The process disclosed herein comprises reacting racemic cis milnacipran with low cost and commercially available resolving agent in a solvent to obtain optical isomers of cis milnacipran with higher yields and having excellent optical purity avoiding multiple crystallizations. The present invention also involves the concept of green chemistry as the invention works well with water as a solvent thereby minimizing the use of any other solvent.

### DETAILED DESCRIPTION OF THE INVENTION

Reference will now be made in detail to the preferred embodiments of the invention. This invention may, however, be embodied in many different forms and should not be construed as limited to the embodiments set forth herein. In addition, and as will be appreciated by one of the skill in the art, the invention may be embodied as a method, system or process.

Efficient, industrially viable and economical process for the resolution of racemic cis milnacipran of formula I is illustrated in the following reaction schemes. The following schemes illustrate the resolution of the compound of formula I or its salts like hydro halide preferably hydrochloride therefrom.

Compound of formula I and its hydrochloride is known in the art. Present invention relates to an improvement over the processes known in the art to overcome the shortcomings therein in the processes disclosed in the prior art.

In first embodiment, racemic cis-milnacipran salt like hydrochloride is converted into corresponding racemic cis milnacipran base by the standard methods by suspending racemic cis milnacipran hydrochloride in a mixture of organic solvent and water followed by the addition of a base under stirring. The organic layer containing cis milnacipran base is separated. Solvent is removed by distillation under reduced pressure.

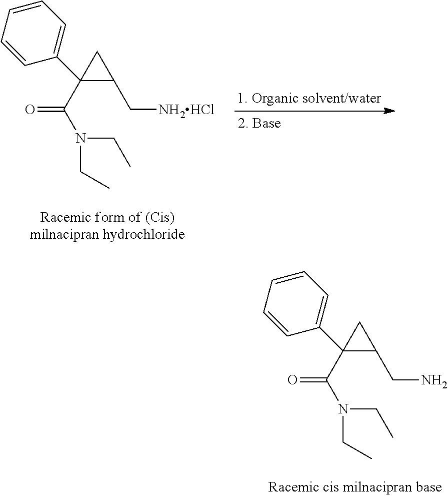

Solvent used in the first embodiment is selected from the water immiscible solvent selected from the group comprising halogenated solvents like dichloromethane, dichloroethane, aromatic solvent like toluene, ester solvent like ethyl acetate, or mixture thereof. Preferably halogenated solvents more preferably dichloromethane (MDC) is used.

In second embodiment racemic cis-milnacipran base is dissolved in a solvent till a clear solution is obtained. This is followed by the addition of optically pure resolving agent of formula III preferably when R is H (it is mandelic acid) under the stirring, heat optionally till cis milnacipran resolvate is precipitated completely. The optically pure resolving agent salt of cis-milnacipran so obtained is filtered off and washed with solvent.

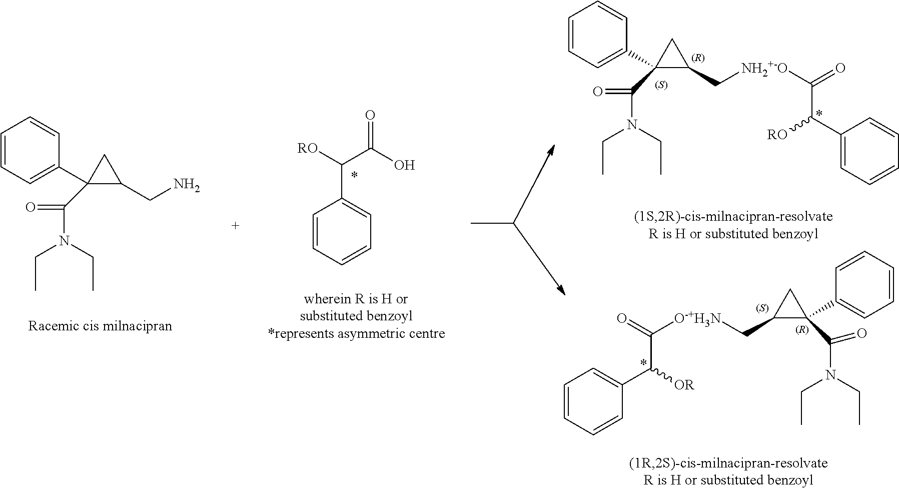

In specific embodiments of the present invention optically active enantiomers of mandelic acid are used as resolving agents for the resolution of racemic cis milnacipran as depicted in the following schematic representations.

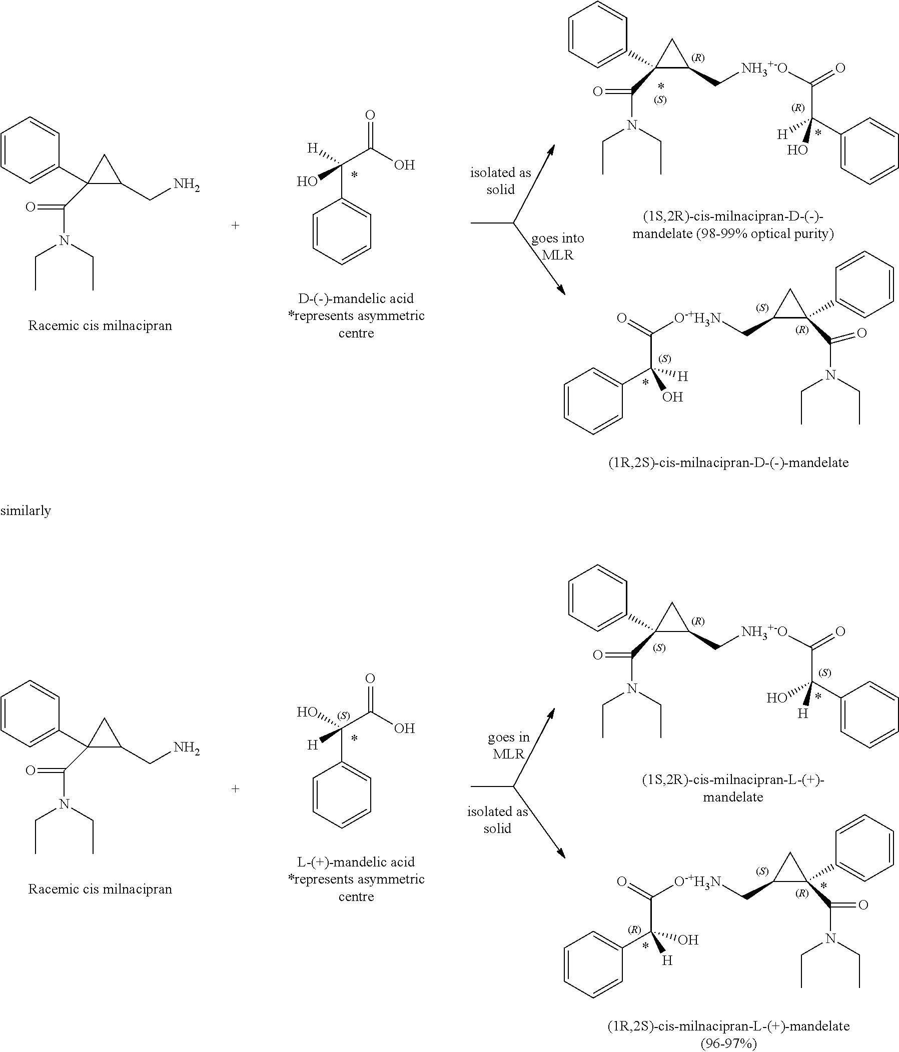

The term MLR refers to mother liqiuor.

Process is characterized in by the fact that when racemic cis milnacipran is resolved by respective mandelic acid and their derivatives, cis milnacipran mandelate with required configuration is isolated as solid while cis milnacipran with other configuration goes into mother liquor hereinafter referred as MLR.

Solvent used in the second embodiment of salt preparation is selected from the group comprising water, aromatic solvent like toluene, alcoholic solvent like isopropanol, ketonic solvent like acetone, ether like methyl tert butyl ether, ester solvent ethyl acetate, and alicyclic solvent like cyclohexane or mixture thereof. Preferably solvent is selected from the group containing water, toluene, acetone, isopropanol, ethyl acetate and mixture thereof. More preferably solvent is water.

The resolving agent is selected from mandelic acid, mandelic acid derivatives and the like. Preferably resolving agent is mandelic acid. Said derivatives of the mandelic acid are selected from the compounds of formula III:

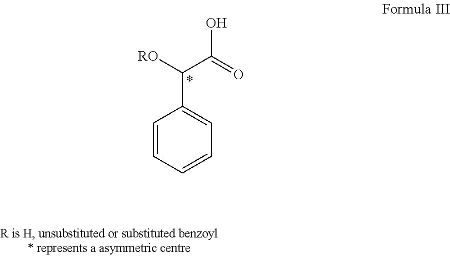

In third embodiment of the present invention, racemic cis milnacipran is dissolved in solvent till it provides a clear solution followed by the addition of aqueous solution of D-(−)-resolving agent of formula III preferably when R is H under stirring, optionally heat till (1S,2R)-cis-milnacipran D-(−)-resolvate is precipitated completely. The optically pure (1S,2R)-cis-milnacipran D-(−)-resolvate so obtained is filtered off and washed with water. This isolated (1S,2R)-cis-milnacipran D-(−)-resolvate contained (1S,2R)-cis-milnacipran with optical purity of about 98% with yield of about 89%.

The resolving agent is selected from D-(−)-mandelic acid, D-(−)-mandelic acid derivatives and the like. Preferably resolving agent is D-(−)-mandelic acid.

In a specific embodiment of the present invention, racemic cis milnacipran is dissolved in water, till a clear solution is obtained followed by the addition of aqueous solution of D-(−)-mandelic acid under stirring, heat optionally, continue stirring till (1S,2R)-cis-milnacipran D-(−)-mandelate is precipitated completely. The optically pure (1S,2R)-cis-milnacipran D-(−)-mandelate so obtained is filtered off and washed with water. This isolated (1S,2R)-cis-milnacipran D-(−)-mandelate contained about 99% optically pure (1S,2R)-cis-milnacipran with yield of about 89%. Only single crystallization from ethyl acetate enhanced optical purity to about 99.9% with yield about 77%.

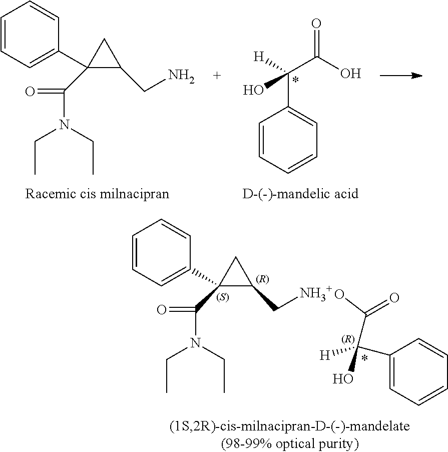

* represents asymmetric centre

(1S,2R)-cis-milnacipran-D-(−)-mandelate is a novel compound and a key intermediate for the preparation of optically pure (1S,2R)-cis-milnacipran.

The melting range of the resolved salt (1S,2R)-cis-milnacipran-D-(−) mandelate is about 117 to about 118° C.

Specific optical rotation of the resolved (1S,2R)-cis-milnacipran-D-(−) mandelate: _{D} [α]²⁵=+13.15° (C=0.95, CHCl₃)

¹H-NMR, Varian, 400 MHz, (CDCl₃), 6 values in ppm: 0.357 (3H, t), 1.15 (3H, t), 1.1518 (1H, s), 1.827 (2H, s), 3.094 (2H, m), 3.336 (3H, m), 3.562 (1H, m), 5.028 (1H, s), 7.351 (10H, m)

C¹³NMR, Varian, 400 MHz, (CDCl₃), 6 values in ppm: 11.43, 11.87, 18.71, 24.16, 34.47, 39.99, 41.56, 42.7, 74.92, 125.74, 126.99, 127.36, 128.08, 128.7, 129.12, 138.81, 140.50, 171, 171.25, 179.29

In another specific embodiment of the present invention, racemic cis milnacipran is dissolved in water, till it provides a clear solution followed by the addition of aqueous solution of L-(+)-mandelic acid under stirring, heat optionally, continue stirring till (1R,2S)-cis-milnacipran L-(+)-mandelate is precipitated completely. The optically pure (1R,2S)-cis-milnacipran L-(+)-mandelate so obtained is filtered off and washed with water. This isolated (1R,2S)-cis-milnacipran L-(+)-mandelate contained about 96% pure (1R,2S)-cis-milnacipran with yield of about 68%.

The resolving agent is selected from L-(+)-mandelic acid, L-(+)-mandelic acid derivatives and the like. Preferably resolving agent is L-(+)-mandelic acid.

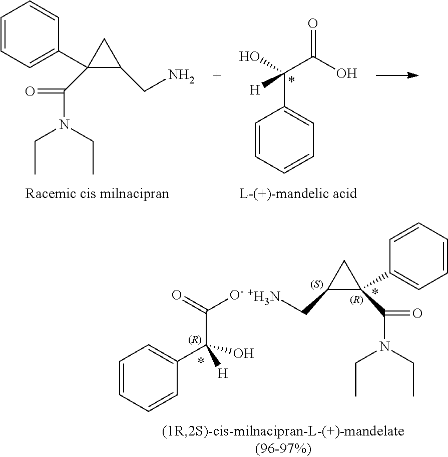

(1R,2S)-cis-milnacipran-L-(+)-mandelate is novel compound and a key intermediate to obtain optically pure (1R,2S)-cis-milnacipran that can be used as reference standard and reference marker for the analytical development studies during the quantitative analysis of (1S,2R)— cis-milnacipran and salts thereof.

The melting range of the resolved salt (1R,2S)-cis-milnaciprari-L-(+) mandelate is about e 114 to about 115° C.

Specific optical rotation of the resolved (1R,2S)-cis-milnacipran-L-(+) mandelate: _{D}[α]²⁵=−13.15° (C=0.95, CHCl₃)

¹H-NMR, Varian, 400 MHz, (CDCl₃), 6 values in ppm: 0.857 (3H, t), 1.156 (3H, t), 1.518 (1H, s), 1.803 (2H, s), 3.094 (2H, m), 3.336 (3H, m), 3.561 (1H, m), 5.029 (1H, s) and 7.350 (10H, m)

C¹³NMR Varian, 400 MHz, (CDCl₃), 6 values in ppm: 11.46, 11.86, 18. 70, 24.16, 34.56, 39.99, 41.56, 42.70, 74.92, 125.74, 126.99, 127.36, 128.99, 128.79, 129.17, 138.80, 140.49, 171.26 and 179.29

In fourth embodiment resolved intermediate novel product so obtained is suspended in the mixture of organic solvent and water followed by the addition of base under stirring till it gets basified. Organic layer is separated washed with water till it becomes neutral, dried over sodium sulphate and solvent is distilled off under the reduced pressure to isolate substantially optically pure milnacipran.

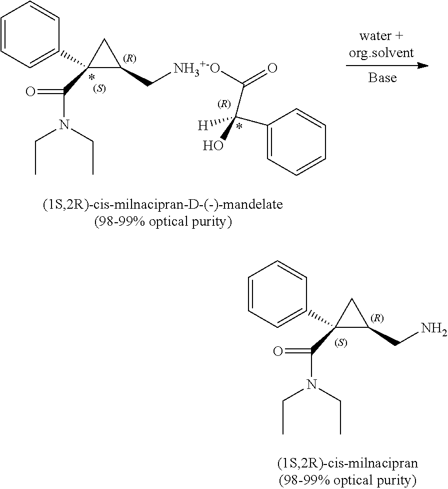

similarly

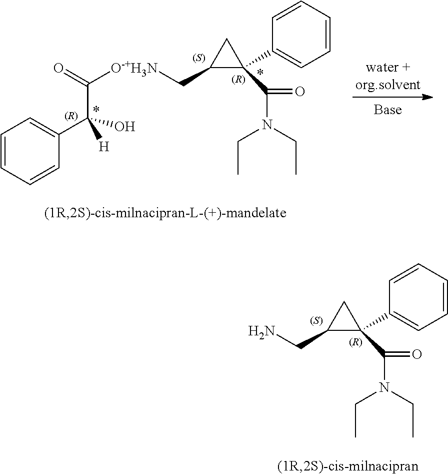

Organic solvent used in fourth embodiment is selected from water immiscible solvent selected from the group comprising halogenated solvents like dichloromethane, dichloroethane, aromatic solvent like toluene, ester solvent like ethyl acetate, and the mixture thereof.

Base used in fourth embodiment is selected from organic amine bases selected from triethyl amine, diethyl amine or inorganic base like ammonia, alkali metal hydroxides, bicarbonates of alkali metal, and carbonates of alkali metals or mixture thereof. Preferably base is selected from alkali metal hydroxides. More preferably base is sodium hydroxide.

In a specific embodiment of the present invention optically pure (1S,2R)-cis-milnacipran D-(−)-mandelate is suspended in the mixture of methylene dichloride (MDC) and water followed by the addition of aqueous 10% sodium hydroxide under stirring till it gets basified till the pH of 8 or above. MDC layer is separated and washed with water till it becomes neutral, dried over sodium sulphate and MDC is distilled off under the reduced pressure to isolate optically pure dextrogyral (1S,2R)-cis-milnacipran base with optical purity of about 98%.

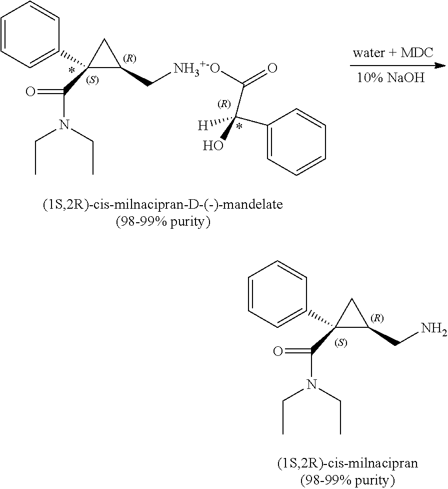

In another specific embodiment of the present invention optically pure (1R,2S)-cis-milnacipran-L-(+)-mandelate is suspended in the mixture of dichloromethane (MDC) and water followed by the addition of aqueous 10% sodium hydroxide under stirring till it gets basified till the pH 8 or above. MDC layer is separated and washed with water till it becomes neutral, dried over sodium sulphate and MDC is distilled off under the reduced pressure to isolate optically pure levogyral (1R,2S)-cis-milnacipran base with optical purity of 98.9%.

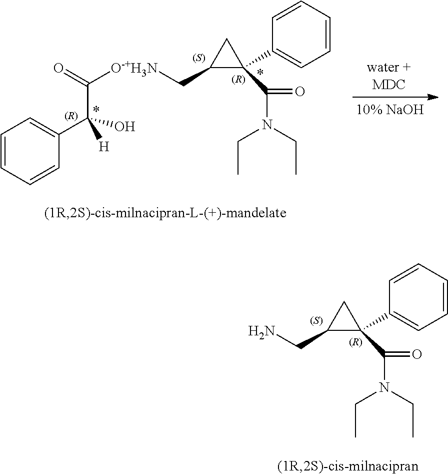

In fifth embodiment optically pure free milnacipran base is further converted into its hydrochloride by taking the free base into alcoholic solvent and dry HCl is passed till pH reaches to about 3 or by treating with isopropanol saturated with HCl.

In a specific embodiment of the present invention optically pure dextrogyral (1S,2R)-cis-milnacipran is further converted into its corresponding hydrochloride salt by taking the free base into isopropanol and dry HCl is passed till pH reaches to about 3 or by reacting pure dextrogyral (1S,2R)-milnacipran with isopropanol saturated with HCl.

Melting range of (1S,2R)-cis-milnacipran hydrochloride is about 190 about 195° C.

Specific optical rotation: D[α]²⁵=+85.72° (C=0.95, Chloroform) at the wave length of 589 nm.

IR spectroscopy (KBr) in cm⁻¹: 2936, 2966, 3061, 3144, 734.9, 1149, 1449, 1449.6, 1612

¹H NMR, Varian, 400 MHz, (CDCl₃) (6) values in ppm: 0.87 (3H, t), 1.09 (4H, t), 1.81 (1H, m), 1.83 (1H, m), 2.45 (1H, m), 3.35 (4H, m), 3.75 (1H, m), 7.16 (2H, m), 7.18 (1H, m) and 7.26 (2H, m)

C¹³ NMR, Varian, 400 MHz, (CDCl₃) (6) values in ppm: 13.0, 13.47, 19.27, 25.20, 35.02, 41.80, 42.30, 126.53, 127.69, 129.61, 140.45, 170.16

In another specific embodiment of the present invention optically pure dextrogyral (1R,2S)-cis-milnacipran is further converted into its corresponding hydrochloride salt by taking the free base into isopropanol and dry HCl is passed till pH reaches to about 3 or by reacting pure levogyral (1R,2S)-milnacipran with isopropanol saturated with HCl.

Melting range of (1R,2S)-cis-milnacipran hydrochloride is about 180 to about 186° C.

Specific optical rotation: _{D}[α]²⁵=−85.72° (C=0.95, Chloroform) at the wave length of 589 nm.

IR spectroscopy (KBr) in cm⁻¹: 2936, 2966, 3061, 3144, 734.9, 1149, 1449, 1449.6 and 1612

¹H NMR, Varian, 400 MHz, (CDCl₃) (δ) values in ppm: 0.87 (3H, t), 1.1 (4H, t), 1.82 (1H, m), 1.83 (1H, m), 2.43 (1H, m), 3.35 (4H, m), 3.75 (1H, m), 7.16 (2H, m), 7.18 (1H, m), 7.28 (2H, m)

C¹³ NMR, Varian, 400 MHz, (CDCl₃) (6) values in ppm: 12.2, 12.85, 18.35, 25.03, 34.71, 39.66, 42.04, 42.41, 125.8, 127.07, 128.88, 138.69, 170.54

The following non limiting examples are provided to illustrate further the present invention, It will be apparent to those skilled in the art that many modifications, variations and alterations to the present disclosure, both to materials, methods and reaction conditions, may be practiced. All such modifications, variations and alterations are intended to be within the spirit and scope of the present inventions.

### Example 1

### Process for Isolation of Racemic Cis-Milnacipran Freebase

25 g (0.0884 mol) of racemic cis-milnacipran hydrochloride is suspended in the mixture of 125 ml of water and 250 ml of dichloromethane (DCM), and 10% sodium hydroxide aqueous solution is added under stirring at room temperature until the aqueous phase is basic (pH=10.5). The organic phases is separated, the aqueous phase is extracted with three times dichloromethane (150 ml each time), the organic extracts are combined, washed two times with brine, then dried over anhydrous sodium sulfate, filtered and dichloromethane is distilled off under reduced pressure to give 20.5 gm (94% yield) racemic cis-milnacipran free-base.

### Example 2

### Resolution of Racemic Cis-Milnacipran by D (−) Mandelic Acid as Resolving Agent Using Water as Solvent

Racemic cis-milnacipran freebase (20.0 g 0.081 moles) obtained by following the method of example 1 is taken in 100 ml water, the mixture is stirred to get clear solution followed by the addition of D (−) mandelic acid (14.0 g, 0.092 moles) solution made in 100 ml water. The mixture is stirred, solid formation is observed, stirring is continued for 1.0 hour. Contents are heated to 60-65° C. to get clear solution and further maintained for 30 min to 60 min. The mixture is gradually brought to room temperature and maintained under stirring for 8-10 hrs. The crystallized solid i.e. (1S,2R)-cis-milnacipran-(D)-mandelate salt is filtered off. Yield is about 87% of theory and optical purity of required cis-(1S,2R)— milnacipran contained is 99-99.5%.

The melting range of the resolved salt (1S,2R)-cis-milnacipran-D-(−) mandelate is observed to be 117-118° C.

Specific optical rotation of the resolved (1S,2R)-cis-milnacipran-D-(−) mandelate: _{D}[α]²⁵=+13.15° (C=0.95, CHCl₃)

¹H-NMR, Varian, 400 MHz, (CDCl₃) 5 values in ppm: 0.357 (3H, t), 1.15 (3H, t), 1.1518 (1H, s), 1.827 (2H, s), 3.094 (2H, m), 3.336 (3H, m), 3.562 (1H, m), 5.028 (1H, s), 7.351 (10H, m)

C¹³NMR Varian, 400 MHz, (CDCl₃) δ values in ppm: 11.43, 11.87, 18.71, 24.16, 34.47, 39.99, 41.56, 42.7, 74.92, 125.74, 126, 99, 127.36, 128.08, 128.7, 129.12, 138.81, 140.50, 171, 171.25, 179.29

### Example 3

### Resolution of Racemic Cis-Milnacipran by D (−) Mandelic Acid as Resolving Agent Using Toluene as Solvent

Racemic cis-milnacipran free base (20.0 g 0.081 moles) in Example-1 is taken in 100 ml toluene under stirring to get clear solution. D (−) mandelic acid (14.0 g, 0.092 moles) is added in one lot. The mixture is stirred for a short time followed by heating the contents at 45-50° C. to get clear solution and maintained it for 60 min. The mixture gradually cooled to room temperature (30-35° C.) and stirring is maintained for overnight (20-24 hr). Filter the crystallized solid obtained i.e. (1S,2R)-cis-milnacipran-(D)-mandelate salt to yield about 71% of theory and optical purity of required (1S,2R)-cis-milnacipran contained in salt is 94-95%. The cake is further washed with hot toluene which results into enhanced optical purity of 98.5-99%.

### Example 4

### Resolution of Racemic Cis-Milnacipran by D (−) Mandelic Acid as Resolving Agent in Ethyl-Acetate and MTBE Mixture

The racemic cis-milnacipran freebase (20.0 g 0.081 moles) following the method of example 1 is taken into 400 ml ethyl-acetate-MTBE under stirring to get clear solution followed by the addition of D (−) mandelic acid (14.0 g, 0.092 moles) in one lot. The mixture is stirred for some time. Solid formation is observed; stirring is continued for 2-3 hours and then heated to 60-65° C. to get clear solution. The temperature is further maintained for 30 min to 60 min. The mixture is gradually cooled to room temperature and maintained under stirring for 2-3 hrs followed by cooling to 10-15° C. for one hour. Crystallized solid i.e. (1S,2R)-cis-milnacipran-(D)-mandelate salt is filtered off. Yield is about 65% of theory and optical purity of required (1S,2R)-cis-milnacipran contained in salt 90-92%.

### Example 5

Resolution of Cis-Racemic Milnacipran by D (−) Mandelic Acid as Resolving Agent Ethyl-Acetate and Water Mixture

Racemic cis-milnacipran freebase (20.0 g 0.081 moles) obtained by following the method of example 1 is taken into 300 ml Ethyl acetate and mixture is stirred to get clear solution followed by the addition of D (−) mandelic acid (14.0 g, 0.092 moles) in one lot and 2.0% water w. r. t. ethyl acetate is added to the above contents. Mixture is stirred for a sometime. Solid formation is observed, stirring is continued for 2-3 hours. Contents are heated to 60-65° C. to get clear solution and maintained it for 30 min to 60 minutes, the mixture is gradually cooled to room temperature and further maintained under stirring for 4-5 hrs then cooled it to 10-15° C. for one hour. The crystallized solid i.e. (1S,2R)-cis-milnacipran mandelate salt is filtered off. Yield is about 70% of theory and optical purity of required (1S,2R)-cis-milnacipran is observed to be 95-97%.

### Example 6

### Formation of (1S,2R)-cis-milnacipran base

10 g (0.025 μmoles) of the resolved product obtained by (1S,2R)-cis-milnacipran-D-(−) mandelate is suspended in the mixture of 100 ml of water and 100 ml of dichloromethane, mixed thoroughly, and 10% sodium hydroxide aqueous solution is added under stirring until the aqueous phase is basic (pH=10.5). The organic phases is separated, the aqueous phase is extracted with dichloromethane (50 ml every time) three times, the organic extracts are combined, washed two times with saturated solution of sodium chloride, then dried with anhydrous sodium sulfate, filtered and evaporated to dryness. The free base of (1S,2R)-cis-milnacipran with optical purity of 99% is afforded, 94% yield (5.8 gm. 0.0235 mol).

### Example 7

### Formation of (1S,2R)-cis-milnacipran hydrochloride

(5.8 gm. 0.0235 mol) the resolved free (1S,2R)-cis-milnacipran base as obtained in example 6 is dissolved in Isopropyl alcohol, the mixture is adjusted to pH 1.5 by the solution of isopropyl alcohol in hydrogen chloride, evaporated under reduced pressure to get concentrated, then n-heptane is added and the mass is precipitated, which is kept under chilling for 2-3 hrs, filtered and dried under vacuum 5.8 g of (1S,2R)-cis-milnacipran hydrochloride, 87% yield. The (1S,2R)-cis-milnacipran hydrochloride so obtained is further confirmed by Infrared spectroscopy and PMR. It is further analyzed for parameters like optical purity, meting point.

IR spectroscopy (KBr): 735.0 (mono-substituted by benzene ring) 1148.1 (tertiary amine) 1465.9 (—CH3) 1614.9 (—CO(NH)—) 1637.8 (bending vibration of —NH2) 2936.2 (—CH2-), 2977.6 (cy-clopropane), 3010.6 (benzene ring), 3400.2 (—NH₂). HR-MS (EJ) calculated for C₁₅H₂₂N₂O 246.32, found 246.1 (free alkali).

¹H-NMR, Varian, 400 MHz, (CDCL₃) δ values in ppm: 0.893 (3H, t) 1.103 (4H, t), 1.749 (1H, m), 1.844 (1H, m), 2.453 (1H, m), 3.354 (4H, m), 3.736 m), 7.189 (2H, m), 7.182 (1H, m).

¹³C-NMR, Varian, 400 MHz, (CDCL₃) δ values in ppm 12.932, 12.179, 17.986, 25.360, 34.647, 42.956, 39.557, 41929, 125.707, 127.151, 128.868, 138.267, 170.583.

Specific optical rotation: [α]_{D} 25=+85.72 (C=0.95, CHC13),

Melting range: 194.29° C.

Optical purity: 99.5-99.8%.

### Example 8

### Resolution of racemic cis-milnacipran by L (+) mandelic acid resolving agent in ethyl acetate

Racemic cis-milnacipran free base (20.0 g 0.081 moles) obtained by following the method of example 1 is taken into 200 ml MTBE. Mixture is stirred to get clear solution followed by the addition of L-(+)-mandelic acid (14.0 g, 0.092 moles). After 10 min, salt came out which is filtered off and recrystallized using 700 ml of ethyl acetate. Filter the recrystallized solid i.e. (1R,2S)-cis-milnacipran-L-(+)-mandelate salt optical purity of required (1R,2S) milnacipran contained in which 98%. Salt is characterized by proton magnetic resolution and C¹³NMR.

Melting range of the resolved crystalline salt (−)-cis-milnacipran-L-(+)-mandelate is observed to be 114-115° C.

¹H-NMR, Varian, 400 MHz, (CDCL₃) δ values in ppm: 0.857 (3H, t), 1.156 (3H, t), 1.518 (1H, s), 1.803 (2H, s), 3.094 (2H, m), 3.336 (3H, m), 3.561 (1H, m), 5.029 (1H, s) and 7.350 (10H, m)

C¹³NMR Varian, 400 MHz, (CDCL₃) δ values in ppm: 11.46, 11.86, 18, 70, 24.16, 34.56, 39.99, 41.56, 42.70, 74.92, 125.74, 126.99, 127.36, 128.99, 128.79, 129.17, 138.80, 140.49, 171.26 and 179.29

### Example 9

Formation of (1R,2S)-cis-milnacipran base:

10 g of the resolved product (1R,2S)-cis-milnacipran-L(+)-Mandelate is suspended in the mixture of 100 ml of water and 100 ml of dichloromethane, mixed thoroughly, and 10% sodium hydroxide aqueous solution is added under stirring until the aqueous phase is basic (pH=11). The organic phases is separated, the aqueous phase is extracted with dichloromethane (50 ml every time) three times, the organic extracts are combined, washed two times with saturated solution of sodium chloride, then dried with anhydrous sodium sulfate, filtered and evaporated to dryness. The free base of (1R,2S)-cis-milnacipran is afforded, 69% yield.

The melting range of the resolved salt (1R,2S)-cis-milnacipran-L-(+) mandelate is observed to be 114-115° C.

Specific optical rotation of the resolved (1R,2S)-cis-milnacipran-L-(+) mandelate: _{D}[α]²⁵=−13.15° (C=0.95, CHCl₃)

¹H-NMR, Varian, 400 MHz, (CDCl₃), δ values in ppm: 0.857 (3H, t), 1.156 (3H, t), 1.518 (1H, s), 1.803 (2H, s), 3.094 (2H, m), 3.336 (3H, m), 3.561 (1H, m), 5.029 (1H, s) and 7.350 (10H, m)

C¹³NMR Varian, 400 MHz, (CDCl₃), δ values in ppm: 11.46, 11.86, 18. 70, 24.16, 34.56, 39.99, 41.56, 42.70, 74.92, 125.74, 126.99, 127.36, 128.99, 128.79, 129.17, 138.80, 140.49, 171.26 and 179.29.

### Example 10

### Formation of (1R,2S)-cis-milnacipran hydrochloride

The resolved free (1R,2S)-cis-milnacipran base as obtained in example is dissolved in Isopropyl alcohol, the mixture is adjusted to pH-3 by the solution of isopropyl alcohol in hydrogen chloride, evaporated to give remainder whose weight is 2-3 times the weight of the free base under reduced pressure, then diisopropyl ether is added and The mass is precipitated, which is kept under chilling for overnight, filtered and dried under vacuum 3.0 g of (1R,2S)-cis-milnacipran hydrochloride, 87% yield of salt formation. The (1R,2S) milnacipran hydrochloride so obtained is further confirmed by Infrared spectroscopy and PMR. It is further analyzed for parameters like optical purity, meting point,

Optical purity of (1R,2S)-cis-milnacipran hydrochloride is observed to be 96%.

Melting range of (1R,2S)-cis-milnacipran hydrochloride is observed to be: 180-186° C.

Specific optical rotation: _{D}[α]²⁵=−85.72° (C=0.95, Chloroform) at the wave length of 589 nm.

IR spectroscopy (KBr) in cm⁻¹: 2936, 2966, 3061, 3144, 734.9, 1149, 1449, 1449.6 and 1612

¹H-NMR, Varian, 400 MHz, (CDCL₃) δ values in ppm: 0.87 (3H, t), 1.1 (4H, t), 1.82 (1H, m), 1.83 (1H, m), 2.43 (1H, m), 3.35 (4H, m), 3.75 (1H, m), 7.16 (2H, m), 7.18 (1H, m), 7.28 (2H, m)

C¹³ NMR, Varian, 400 MHz, (CDCL₃) δ values in ppm: 12.2, 12.85, 18.35, 25.03, 34.71, 39.66, 42.04, 42.41, 125.8, 127.07, 128.88, 138.69, 170.54

1. A process for preparing optically pure milnacipran and their pharmaceutically acceptable salts comprising the steps of:
a) dissolving racemic cis milnacipran of the formula I in a solvent; 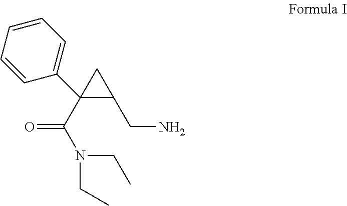
b) adding resolving agent of formula III, optionally dissolved in the solvent to the solution of racemic cis milnacipran obtained in step (a), comprising optional heating to obtain a clear solution; 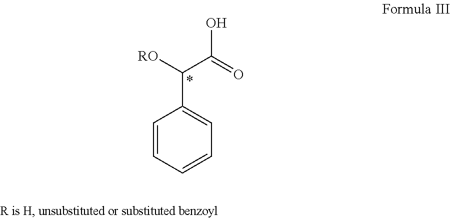
* represents asymmetric centre
c) cooling reaction mass of step (b) to separate optically pure cis-milnacipran resolvate salt wherein said salt is filtered and washed with the solvent;
d) dissolving the cis-milnacipran resolvate salt obtained in step (c) in the solvent to obtain a solution and adding about 10% base to said solution to obtain optically pure cis-milnacipran.
e) optionally converting said optically pure cis-milnacipran into pharmaceutically acceptable salts.

2. The process of claim 1, wherein cis milnacipran base of formula I is contacted with D-(−) mandelic acid in the solvent to obtain optically pure dextrogyral cis-milnacipran.

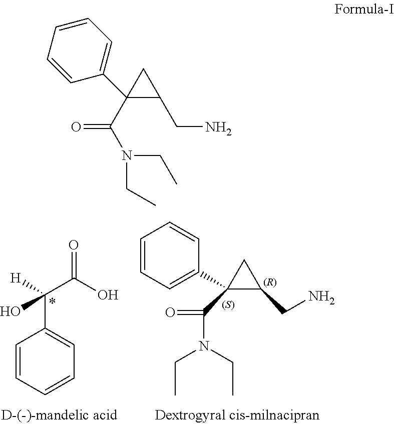

3. The process of claim 1, wherein cis milnacipran base of formula I is contacted with L-(+) mandelic acid in the solvent to obtain optically pure levogyral cis-milnacipran.

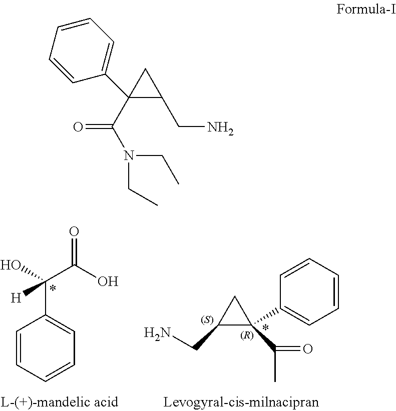

4. The process of claim 1, wherein the solvent is selected from the group comprising water, aromatic hydrocarbons, C1-C7 alcohols, aliphatic ketones, ethers, esters, aliphatic acyclic and acyclic hydrocarbons, halogenated hydrocarbons and mixtures thereof.

5. The process of claim 1, wherein the solvent is water.

6. The process of claim 1, wherein cis milnacipran base of formula I is contacted with D-(−) mandelic acid in water to obtain optically pure dextrogyral cis milnacipran.

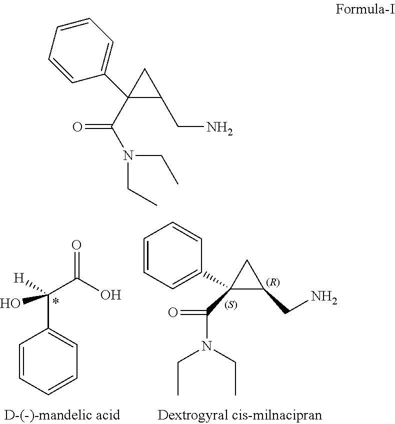

7. The process of claim 1, wherein cis milnacipran base of formula I is contacted with L-(+) mandelic acid in water to obtain optically pure levogyral cis-milnacipran.

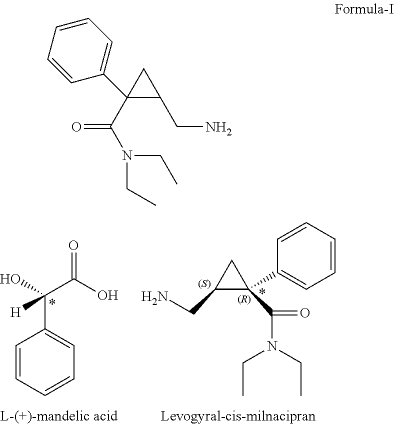

8. A novel and crystalline compound of the formula A

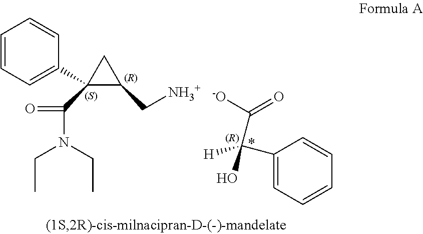

9. A novel and crystalline compound of the formula B [chemical structure]
